(19) United States
(12) Patent Application Publication
(10) Pub. No.: US 2020/0181552 A1
(43) Pub. Date: Jun. 11, 2020

(54) HANDLING FEATURES FOR MICROCAVITY CELL CULTURE VESSEL

(71) Applicant: CORNING INCORPORATED, Corning, NY (US)

(72) Inventors: Gregory Roger Martin, Acton, ME (US); Allison Jean Tanner, Portsmouth, NH (US)

(21) Appl. No.: 16/628,492

(22) PCT Filed: Jul. 13, 2018
(86) PCT No.: PCT/US2018/042145

§ 371 Date: Jan. 3, 2020

Related U.S. Application Data
(60) Provisional application No. 62/532,648, filed on Jul. 14, 2017.

(51) Int. Cl. C12M 1/32; C12M 1/24; C12M 1/00

(52) U.S. Cl. CPC C12M 23/12; C12M 23/38; C12M 23/08

(57) ABSTRACT

The disclosure provides a cell culture vessel (100) having a surface (316) for culturing cells that has a microcavity array (115), suitable for culturing cells in 3D, either integrally provided in the bottom surface of the vessel, or provided by an insert (216) having an array of microcavities, placed on or affixed to the bottom surface of the vessel. The disclosure provides baffles (113) in the cell culture chamber and dams (130) in the neck of the vessel which control the flow of liquid into and out of microcavities to allow the microcavities to be filled and emptied with minimal turbulence, thus creating less disturbance for spheroids resting in the microcavities.

### CROSS REFERENCE TO RELATED APPLICATION

The application claims the benefit of priority of U.S. Provisional Application Ser. No. 62/532,648 filed on Jul. 14, 2017, entitled “Cell Culture Container and Methods of Culturing Cells”, the content of which is relied upon and incorporated herein by reference in its entirety.

### FIELD

The present disclosure relates generally to a cell culture vessel and methods of culturing cells, and more particularly, to a cell culture vessel for containing and manipulating three-dimensional cells and methods of culturing three-dimensional cells in the cell culture vessel.

### BACKGROUND

It is known to contain and culture three-dimensional cells in a cell culture vessel.

### SUMMARY

The following presents a simplified summary of the disclosure in order to provide a basic understanding of some exemplary embodiments described in the detailed description.

In embodiments, the disclosure provides a cell culture vessel having a necked opening, a cell culture chamber, a top, a bottom, sidewalls, an endwall and a surface for culturing cells that has a microcavity array, either integrally provided in the bottom surface of the vessel, or provided by an insert, having an array of microcavities, placed on or affixed to the bottom surface of the vessel. In embodiments, the array of microcavities does not extend along the entire length of the cell culture chamber of the vessel. In embodiments, the array of microcavities extends less than the entire length of the cell culture chamber (Lc). In embodiments, the array of microcavities extends a length (Lᵢ). Lᵢ is less than Lc. Between the array of microcavities and the end wall of the vessel, in embodiments, is a baffle. The baffle has a length (L_{b}). The baffle occupies the space between the endwall of the vessel and the microcavity array. Lᵢ+Lb=Lc. In embodiments, the baffle defines a reservoir when the vessel is placed with the necked opening up. The baffle controls the flow of liquid into microcavities to allow the microcavities to be filled with media with minimal turbulence, thus creating less disturbance for spheroids resting in the microcavities. In embodiments, baffles may be angled, curved, square or any shape. In additional embodiments, and also to reduce turbulence created by the movement of liquid into or out of the vessel, the necked opening of the vessel may have dams which interrupt the flow of liquid, such as liquid media, entering or exiting the vessel. In embodiments the dams may be square or curved or any shape. These features may be present alone or in combination. For example, the vessel may have a microcavity array and a dam that is curved or straight. The vessel may have a microcavity array and a baffle and the baffle may be angled or square. The vessel may have a microcavity array and a dam and a baffle. And, the dam may be curved or square and the baffle may be angled or square. Methods for culturing cells, introducing media and removing media from the vessels are also provided.

The above embodiments are exemplary and can be provided alone or in any combination with any one or more embodiments provided herein without departing from the scope of the disclosure. Moreover, it is to be understood that both the foregoing general description and the following detailed description present embodiments of the present disclosure, and are intended to provide an overview or framework for understanding the nature and character of the embodiments as they are described and claimed. The accompanying drawings are included to provide a further understanding of the embodiments, and are incorporated into and constitute a part of this specification. The drawings illustrate various embodiments of the disclosure, and together with the description, serve to explain the principles and operations thereof.

### BRIEF DESCRIPTION OF THE DRAWINGS

These and other features, embodiments, and advantages of the present disclosure can be further understood when read with reference to the accompanying drawings in which:

FIG. 1 schematically illustrates a side view of a first exemplary cell culture vessel in accordance with embodiments of the disclosure;

FIG. 2 shows a plan view of the first exemplary cell culture vessel along line 2-2 of FIG. 1 in accordance with embodiments of the disclosure;

FIG. 3 shows a cross-sectional view of the first exemplary cell culture vessel along line 3-3 of FIG. 2 in accordance with embodiments of the disclosure;

FIG. 4 shows a cross-sectional view of the first exemplary cell culture vessel along line 4-4 of FIG. 1 in accordance with embodiments of the disclosure;

FIG. 5 illustrates an enlarged schematic representation of an exemplary embodiment of a portion of the first exemplary cell culture vessel taken at view 5 of FIG. 4 including a surface having a microcavity array including a plurality of microcavities in accordance with embodiments of the disclosure;

FIG. 6 shows a cross-sectional view of the portion of the first exemplary cell culture vessel including a surface having a microcavity array including a plurality of microcavities along line 6-6 of FIG. 5 in accordance with embodiments of the disclosure;

FIG. 7 shows an alternative exemplary embodiment of the cross-sectional view of the portion of the first exemplary cell culture vessel including a surface having a microcavity array including a plurality of microcavities of FIG. 6 in accordance with embodiments of the disclosure;

FIG. 8 shows an exemplary embodiment of a partial cross-sectional view of a portion of the first exemplary cell culture vessel along line 8-8 of FIG. 2 including a stepped profile in accordance with embodiments of the disclosure;

FIG. 9 shows an alternative exemplary embodiment of the partial cross-sectional view of the portion of the first exemplary cell culture vessel of FIG. 8 including an inclined profile in accordance with embodiments of the disclosure;

FIG. 10 shows an exemplary embodiment of a partial cross-sectional view of a portion of the first exemplary cell culture vessel along line 10-10 of FIG. 1 including a baffle including a convex profile in accordance with embodiments of the disclosure;

FIG. 11 shows an alternative exemplary embodiment of the partial cross-sectional view of the portion of the first exemplary cell culture vessel of FIG. 10 including a baffle including a concave profile in accordance with embodiments of the disclosure;

FIG. 12 shows an alternative exemplary embodiment of the cross-sectional view of the first exemplary cell culture vessel of FIG. 3 including a method of culturing cells in the first exemplary cell culture vessel in accordance with embodiments of the disclosure;

FIG. 13 shows an exemplary step of the method of culturing cells in the first exemplary cell culture vessel of FIG. 12 in accordance with embodiments of the disclosure;

FIG. 14 illustrates an enlarged schematic representation of an exemplary embodiment of a portion of the first exemplary cell culture vessel taken at view 14 of FIG. 13 including a surface having a microcavity array including a plurality of microcavities in accordance with embodiments of the disclosure;

FIG. 15 shows an exemplary step of the method of culturing cells in the first exemplary cell culture vessel of FIG. 13 in accordance with embodiments of the disclosure;

FIG. 16 illustrates an enlarged schematic representation of an exemplary embodiment of a portion of the first exemplary cell culture vessel taken at view 16 of FIG. 17 including a surface having a microcavity array including a plurality of microcavities and a method of culturing cells in at least one microcavity of the plurality of microcavities in accordance with embodiments of the disclosure;

FIG. 17 shows an alternative exemplary embodiment of the partial cross-sectional view of the portion of the first exemplary cell culture vessel of FIG. 10 including a baffle and a method of adding material to the vessel with a dispensing-port in accordance with embodiments of the disclosure;

FIG. 18 shows an alternative exemplary embodiment of the partial cross-sectional view of the portion of the first exemplary cell culture vessel of FIG. 10 including a baffle and a method of removing material from the vessel with a collecting-port in accordance with embodiments of the disclosure;

### DETAILED DESCRIPTION

Features will now be described more fully hereinafter with reference to the accompanying drawings in which exemplary embodiments of the disclosure are shown. Whenever possible, the same reference numerals are used throughout the drawings to refer to the same or like parts. However, this disclosure can be embodied in many different forms and should not be construed as limited to the embodiments set forth herein.

A cell culture vessel (e.g., flask) can provide a sterile chamber for culturing cells. In some embodiments, culturing cells can provide information related to the study of diseases and toxicology, the efficacy of medications and treatments, characteristics of tumors, organisms, genetics, and other scientific, biological, and chemical principles of and relating to cells. As compared to two-dimensional cell cultures, in some embodiments, three-dimensional cell cultures can produce multicellular structures that are more physiologically accurate and that more realistically represent an environment in which cells can exist and grow in real life applications as compared to two-dimensional cell culture. For example, three-dimensional cell cultures have been found to more closely provide a realistic environment simulating “in vivo” (i.e. within the living, real-life) cell growth; whereas two-dimensional cell-cultures have been found to provide an environment simulating “in vitro” (i.e., within the glass, in a laboratory setting) cell growth that is less representative of a real-life environment. By interacting with and observing the properties and behavior of three-dimensional cell cultures, advancements in the understanding of cells relating to, for example, the study of diseases and toxicology, the efficacy of medications and treatments, characteristics of tumors, organisms, genetics, and other scientific, biological, and chemical principles of and relating to cells can be achieved.

In embodiments, the cell culture vessel 100 can include a bottom 108, a top 101, and endwall 107 and sidewalls 106, each having internal surfaces that contact liquid media and cells in culture. These internal surfaces define the cell culture chamber 103. At least one of these surfaces can be more particularly adapted for cell growth. For example, a cell culture surface may be treated with a coating to encourage or discourage cells to stick to a surface. Or, to support the culture of spheroid cells, the cell growth surface can include a plurality of microcavities or compartments (e.g., micron-sized wells, submillimeter-sized wells) arranged, for example, in an array. The cell growth surface can be integral to the flask or can be a separate surface having a microcavity array placed or affixed in the cell growth chamber. The top surface, the bottom surface, one or more side surfaces or a combination of these can include microcavities in an array.

For example, in some embodiments, a single spheroid can form in each microcavity of the plurality of microcavities. Cells introduced into the vessel in liquid media will settle into a microcavity by gravity. One or more cells suspended in liquid media will fall through the liquid and settle within each microcavity. The shape of the microcavity (e.g., a concave surface defining a well), and a surface coating of the microcavity that prevents the cells from attaching to the surface can also facilitate growth of cells into three-dimensional form, forming a spheroid in each microcavity.

Microcavities can be, for example, formed in an undulating or sinusoidal shape forming microcavities or microwells having rounded tops and rounded bottoms. These rounded edges may prevent the formation of bubbles when liquid media fills the vessel. In some embodiments, the flask can be filled with a material (e.g., media, solid, liquid, gas) that facilitates growth of three-dimensional cell cultures (e.g., cell aggregates, organoids or spheroids). For example, a media including cells suspended in a liquid can be added to the cell culture chamber or vessel. The suspended cells can collect in the plurality of microcavities by gravity and can form (e.g., grow) into grouping or cluster of cells. The grouped or clustered cells grow in three dimensions to form cells in 3D, otherwise known as a spheroid or an organoid. A single cluster of cells or spheroid forms in a single microcavity. Thus, a vessel, or a cell culture chamber, having a cell culture surface having an array of microcavities, can be used to culture an array of spheroids, each residing in its own microcavity.

During culturing, the spheroids can consume media (e.g., food, nutrients) and produce metabolites (e.g., waste) as a byproduct. Thus, in some embodiments food in the form of media can be added to the cell culture chamber during culturing and waste media can be removed from the cell culture chamber during culturing. This ability to change the media to feed cells and remove waste products, is important for the long-term culture of cells. However, adding and removing media may create turbulence which may disrupt or displace spheroids resting in microcavities. This is especially true when the microcavities are coated with a low binding coating to prevent the cells from sticking to the microcavity surface. The spheroids are loose (not attached to the surface) and may be dislodged and float free of their microcavity resting place. It is not preferable to dislodge spheroids growing in culture for many reasons. The spheroids may be removed from the culture with the removal of spent media. Dislodged spheroids may settle into occupied microcavities, and may merge with other spheroids to form non-uniform 3D cellular structures. That is, after a media change, some spheroids may be bigger than others in the culture. This reduces the uniformity of the cell culture and may affect results of assays or other tests carried out on 3D cells. In this disclosure, structures are disclosed which reduce turbulence, reducing the risk of displacing spheroids from the microcavities, thus promoting the long-term culture of spheroids.

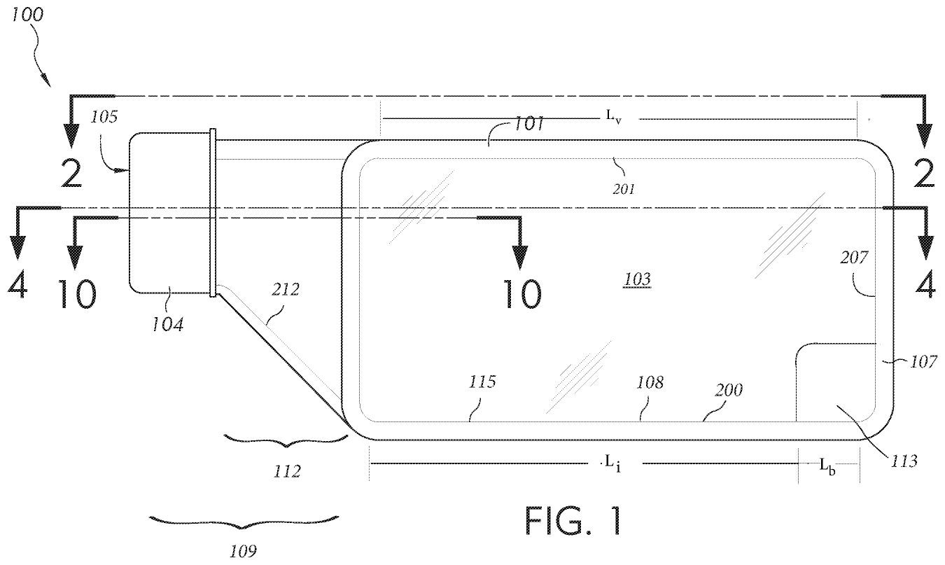

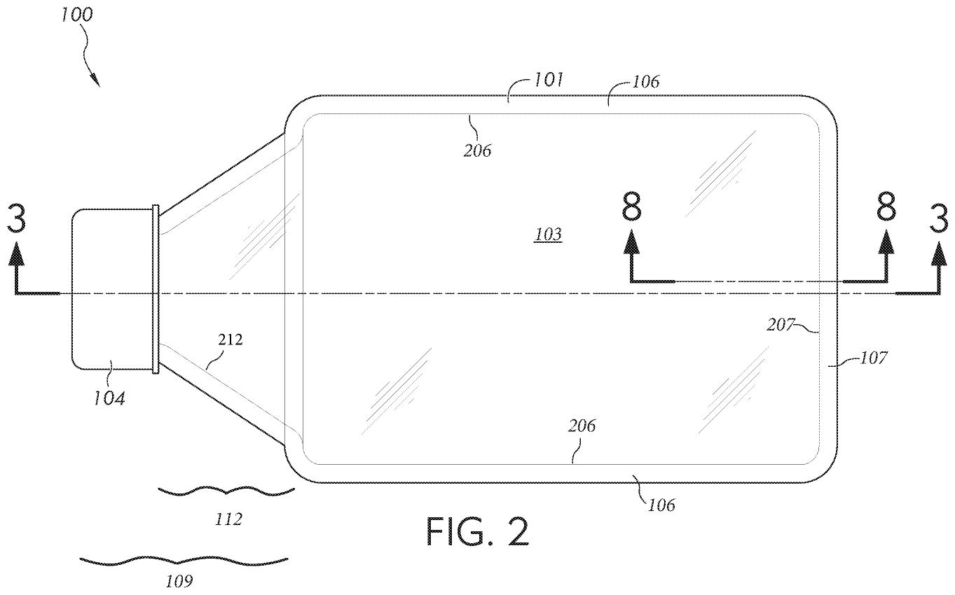

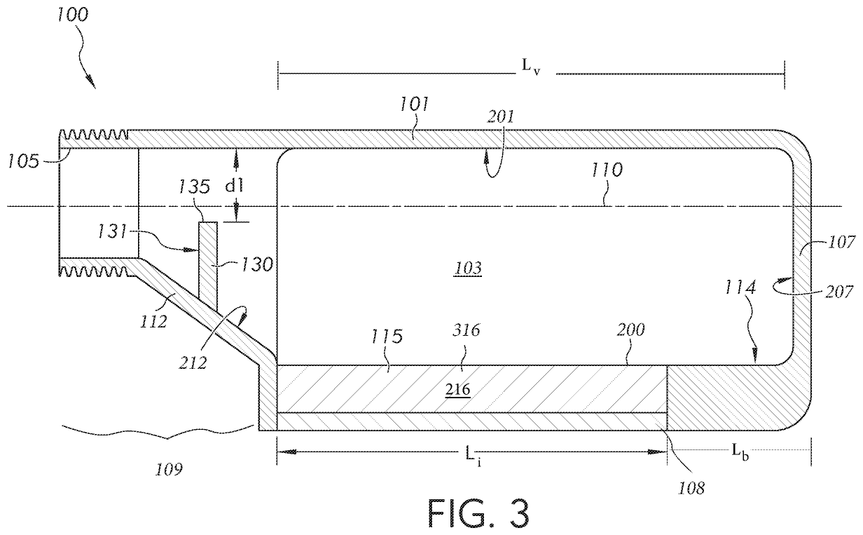

Embodiments of a cell culture vessel 100 and methods of culturing cells in the exemplary cell culture vessel 100 are described with reference to FIGS. 1-18. FIG. 1 schematically illustrates a side view of the exemplary cell culture vessel 100. FIG. 2 is a plain view of the vessel 100 along line 2-2 of FIG. 1. As shown in FIG. 1 and FIG. 2, an embodiment of a cell culture vessel 100 is shown. The cell culture vessel 100 has a port or aperture 105 (shown in FIG. 1 with a cap 104, but see FIG. 3) and a neck 112 connecting the port or aperture 105 to the cell culture chamber 103. In embodiments the aperture can be releasably sealed. For example, in embodiments, the aperture 105 section of the neck 112 can have threads (either interior or exterior) that allow a cap 104 to be releasably sealed 105 by a cap 104 having complimentary threaded structure. Or, the necked opening 105 can be releasably sealed by any other mechanism known in the art to close a vessel. The aperture 105 combined with the neck 112 is the necked opening 109 (See FIG. 3). The necked opening 109 extends through a wall of the cell culture chamber 103 and is in fluid communication with the cell culture chamber 103. The necked opening 113 allows liquid to be introduced and removed from the cell culture chamber (the interior) of the vessel.

The cell culture surface 200 of the vessel 100 is, in embodiments, the bottom 108 of the vessel 100 when the vessel 100 is oriented for cell growth. In embodiments, the vessel 100 is oriented for cell growth when the vessel 100 is placed with the bottom 108 of the vessel 100 flat on a surface. The vessel 100 may also have sidewalls 106 and an endwall 107 opposite the necked opening 109, a top 101 and bottom 108. In embodiments the top 101 is opposite the cell culture surface 200 of the vessel 100. In embodiments, the necked opening 109 is opposite the endwall 107 of the vessel 100. In embodiments, the cell culture surface 200 has a microcavity array 115. Each of these structures (the necked opening 109, the top 101, the bottom 108, the sidewalls 106 and the endwall 107) of the vessel 100 have internal surfaces facing inside the vessel 100. That is, the top 101 has an interior surface 201. The end wall 107 has an interior surface 207. The sidewalls 106 have interior surfaces 206. The neck 112 has an internal surface 212 and embodiments, the bottom 108 has an interior surface. The inside of the vessel is the cell culture chamber 103, the space inside the vessel 100, defined by the top 101, the bottom 108, the sidewalls 106 and the endwall 107 where cells reside inside the vessel 100.

FIG. 2 shows a plan view of the vessel 100 along line 2-2 of FIG. 1. In some embodiments, the cell culture vessel 100 can be manufactured from a material including, but not limited to, polymer, polycarbonate, glass, and plastic. In an embodiment, the vessel 100 is illustrated as being manufactured from a clear (e.g., transparent) material; although, in some embodiments, the vessel 100 may, alternatively, be manufactured from a semi-transparent, semi-opaque, or opaque material without departing from the scope of the disclosure.

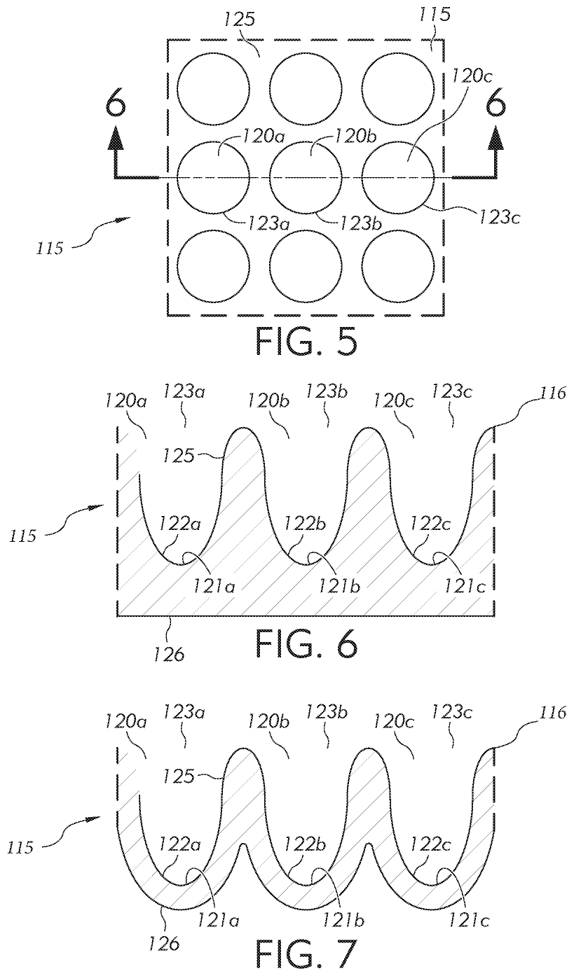

As shown in FIG. 3, which shows a cross-sectional view of the vessel 100 along line 3-3 of FIG. 2, bottom 108 may have an insert 216 resting upon bottom 108. Insert 216 has an inner surface 316. The inner surface 316 of insert 216 may have an array of microcavities 115. In embodiments the microcavities in the array of microcavities 115 are coated with a coating that inhibits cell attachment. Inner surface 316 of insert 216 having an array of microcavities 115 (See FIGS. 5-7), in embodiments, forms the cell culture surface 200. The insert may be of any material suitable for forming a microcavity array 115, including polymer, polycarbonate, glass, and plastic. In embodiments, the insert 216 is placed on bottom 108 during manufacture of the vessel 100. In embodiments, the insert 216 is affixed to bottom 108 during manufacture of the vessel 100, using any methods known in the art including gluing, welding, sonic welding, ultrasonic welding, laser welding or the like.

Turning back to FIG. 1 and FIG. 2, in some embodiments, the vessel 100 can include a cap 104 oriented to cover the port 105 to at least one of seal and block the port 105, thereby obstructing a path into the cell culture chamber 103 from outside the vessel 100 through the port 105. For clarity, the cap 104 is removed and, therefore, not shown in other drawing figures, although it is to be understood that the cap 104 can be provided and selectively added to or removed from the port 105 of the vessel 100, in some embodiments, without departing from the scope of the disclosure. In some embodiments, the cap 104 can include a filter that permits the transfer of gas in to and/or out of the cell culture chamber 103 of the vessel 100. For example, in some embodiments, the cap 104 can include a gas-permeable filter oriented to regulate a pressure of gas within the cell culture chamber 103, thereby preventing pressurization (e.g., over-pressurization) of the cell culture chamber 103 relative to a pressure of the environment (e.g., atmosphere) outside the vessel 100.

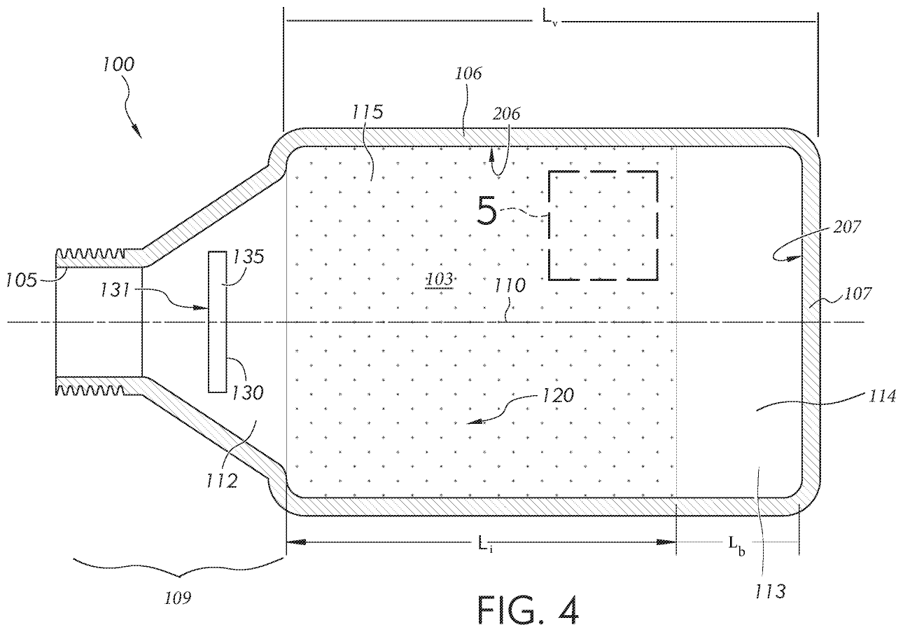

FIG. 4 shows a cross-sectional view along line 4-4 of FIG. 1. In some embodiments, the end wall 107 is positioned opposite the port 105 along an axis 110 of the vessel 100, and the insert 216 having a microcavity array 115 spans a length “Lᵢ” of the cell culture chamber 103. In embodiments the length “Lᵢ” of the insert 216, where there is an insert, or the length of the array of microcavities 115 when the microcavities are provided in the interior surface 208 of the bottom 108 of the cell culture chamber 103, is less than the length of the “L_{c}” of the cell culture chamber 103 of the vessel 100. That is, in embodiments, the array of microcavities 115 does not extend along the entire length of the cell culture chamber of the vessel L_{c}. In embodiments, the array of microcavities extends less than the entire length of the cell culture chamber (L_{c}). In embodiments, the array of microcavities extends a length (Lᵢ). Lᵢ is less than L_{c}. Between the array of microcavities 115 and the end wall 107 of the vessel 100, in embodiments, is a baffle 113. The baffle 113 has a length (L_{b}). The baffle 113 occupies the space between the endwall 107 of the vessel 100 and the microcavity array 115.

Lᵢ+L_{b}=L_{c} .Equation 1:

In embodiments, the baffle defines a reservoir when the vessel is placed with the necked opening up. Along the end wall 107 of the vessel 100, there is a baffle 113 (described in more detail below) having a baffle face 114. FIG. 4 also shows a dam 130 in the necked opening 109 of the vessel 100. In the embodiment shown in FIG. 4, the dam is square, but the dam can be any shape including curved, concave, convex, squiggly, or any other shape. In addition, the profile of the dam may be square, as shown in FIG. 4, or the profile of the dam may be curved, concave, convex, or any other shape.

FIG. 5 shows an enlarged schematic representation of a portion of the surface having a microcavity array 115 taken at view 5 of FIG. 4. Additionally, FIG. 6 shows a cross-sectional view of the portion of the surface having a microcavity array 115 along line 6-6 of FIG. 5, and FIG. 7 shows an alternative embodiment of the cross-sectional view of FIG. 6. As shown in FIGS. 5-7, in some embodiments, each microcavity 120 (shown as 120a, 120b, 120c) in the array of microcavities 115 has an opening 123a, 123b, 123c (e.g., in the interior surface 116 of the array of microcavities 115) at the top of each microcavity 120. And, each microcavity 120 in the array of microcavities 115 can include a concave surface 121a, 121b, 121c (See FIG. 6 and FIG. 7) defining a well 122a, 122b, 122c. Further, each microcavity 120a, 120b, 120c can include a well 122a, 122b, 122c. These structures are present whether the microcavity array 115 is integral to the bottom 108 of the vessel 100 or whether the microcavity array is provided by an insert 216 having a microcavity array 115.

As shown in FIG. 6, in some embodiments, the interior surface 116 of the microcavity array 115 can include a non-linear (e.g., undulating, sinusoidal) profile defining the microcavities 120. The bottom side 126 of the surface having a microcavity array 115 can include a planar (e.g., flat) profile, as shown as 126a in FIG. 6. These structures are present whether the microcavity array is integral to the bottom 108 of the vessel 100 or whether the microcavity array is provided by an insert 216 having a microcavity array 115. Similarly, as shown in FIG. 7, in some embodiments, both the interior surface 116 and the exterior surface 126 of the microcavity array 115 can include a non-planar (e.g., undulating, sinusoidal) profile. These structures are present whether the microcavity array is integral to the bottom 108 of the vessel 100 or whether the microcavity array is provided by an insert 216 having a microcavity array 115. As shown in FIG. 7, when the microcavity array 115 is integral to the bottom 108 of the vessel 100, in embodiments when the profile of the microcavities 120 has a uniform thickness, the interior surface displays an array of microcavities 120 and the bottom side of the surface has an array of microprojections 126b. These are shown in FIG. 7 as 126b. The exterior surface 126 of the bottom 108 of the vessel 100 will exhibit these undulations, and may be “bumpy”. That is, in embodiments, the exterior surface 126 of the bottom 108 of the vessel 100, may show the bottom contour of the individual microcavities 120. As shown in FIG. 7, the bottom contour of the microcavities is the bottom side of the undulating structure of the microcavities, but the microcavities may be in any shape, and therefore the exterior surface of the bottom 108 of the vessel 100 may be in any shape which is the bottom side of the microcavities 120. That is, the exterior surface 126 of the bottom 108 of the vessel 100 may have an array of microprojections or may be “bumpy”. This is true also when the microcavity array is provided by an insert 216. In that case, the exterior surface of the insert may be “bumpy”, while the exterior surface of the bottom 108 of the vessel 100 is planar. That is, the insert 216 may have a bumpy exterior surface 126b, or an exterior surface having an array of microprojections 126b, having an array of microprojections 126b. and it may rest against the interior surface of the bottom 108, which may be smooth.

The surface having a microcavity array 115 shown in FIG. 6 illustrates an interior surface 116 having the microcavity array 115 having an undulating or sinusoidal profile in FIG. 6 that creates an array of microcavities 115. The exterior surface 126a of microcavity array 115 has a planar (e.g., flat) profile. In FIG. 7 where the interior surface 116 and the exterior surface 126 of the microcavity array 115 the bottom surface has an array of rounded microprojections. The profile of the microcavity array 115 shown in FIG. 7 is reduced. Thus, embodiments, a thinner profile of material creating a microcavity array results in a bottom surface having an array of microprojections 126b. This can reduce the amount of material used to make the surface having a microcavity array 115 and can provide a surface having a microcavity array 115 that includes thinner walled microcavities 120a, 120b, 120c than, for example a surface having a microcavity array 115 where the exterior surface 126a of the microcavity array 115 includes a planar (e.g., flat) profile (FIG. 6). In some embodiments, thinner walled microcavities 120a, 120b, 120c can provide a thinner profile of material that allows the walls of the microcavities 120 to be gas permeable. In embodiments, this may permit a higher rate of gas transfer (e.g., permeability) of the surface having a microcavity array to provide more gas in to and out of the wells 122a, 122b, 122c during cell culturing. Thus in some embodiments, providing both the interior surface 116 and the exterior surface 126 of the microcavity array 115 with a non-planar (e.g., undulating, sinusoidal) profile (see, for example FIG. 7) can provide a healthier cell culture environment, thereby improving the culturing of cells in the microcavities 120a, 120b, 120c. In addition, in embodiments, the two profiles shown in FIG. 6 and FIG. 7 may be made using different manufacturing methods. The profile as shown in FIG. 6 may be made by stamping or imprinting or molding a shape into one side of a flat sheet of relatively thick material. The profile shown in FIG. 7 may be made by molding or rolling a thin sheet of material to make the thinner profile shown in FIG. 7.

In some embodiments, the surface having a microcavity array 115 can be a polymeric material including, but not limited to, polystyrene, polymethylmethacrylate, polyvinyl chloride, polycarbonate, polysulfone, polystyrene copolymers, fluoropolymers, polyesters, polyamides, polystyrene butadiene copolymers, fully hydrogenated styrenic polymers, polycarbonate PDMS copolymers, and polyolefins such as polyethylene, polypropylene, polymethyl pentene, polypropylene copolymers and cyclic olefin copolymers. Additionally, in some embodiments, at least a portion of the well 122a, 122b, 122c defined by the concave surface 121a, 121b, 121c can be coated with a low binding material, thereby making the at least a portion of the well 122a, 122b, 122c non-adherent to cells. For example, in some embodiments, one or more of perfluorinated polymers, olefins, agarose, non-ionic hydrogels such as polyacrylamides, polyethers such as polyethyleneoxide, polyols such as polyvinylalcohol or mixtures thereof can be applied to at least a portion of the well 122a, 122b, 122c defined by the concave surface 121a, 121b, 121c.

Moreover, in some embodiments, each microcavity 120a, 120b, 120c of the plurality of microcavities 120 can include a variety of features and variations of those features without departing from the scope of the disclosure. For example, in some embodiments the plurality of microcavities 120 can be arranged in an array (an array of microcavities 115) including a linear array (shown), a diagonal array, a rectangular array, a circular array, a radial array, a hexagonal close-packed arrangement, etc. Additionally, in some embodiments, the opening 123a, 123b, 123c can include a variety of shapes. In some embodiments, the opening 123a, 123b, 123c can include one or more of a circle, an oval, a rectangle, a quadrilateral, a hexagon, and other polygonal shapes. Additionally, in some embodiments, the opening 123a, 123b, 123c can include a dimension (e.g., diameter, width, diagonal of a square or rectangle, etc.) from about 100 microns (μm) to about 5000 μm. For example, in some embodiments, the opening 123a, 123b, 123c can include a dimension of 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm, 1000 μm, 1500 μm, 2000 μm, 2500 μm, 3000 μm, 3500 μm, 4000 μm, 4500 μm, 5000 μm, and any dimension or ranges of dimensions encompassed within the range of from about 100 μm to about 5000 μm.

In some embodiments, the well 122a, 122b, 122c defined by the concave surface 121a, 121b, 121c can include a variety of shapes. In some embodiments, the well 122a, 122b, 122c defined by the concave surface 121a, 121b, 121c can include one or more of a circular, elliptical, parabolic, hyperbolic, chevron, sloped, or other cross-sectional profile shape. Additionally, in some embodiments, a depth of the well 122a, 122b, 122c (e.g., depth from a plane defined by the opening 123a, 123b, 123c to the concave surface 121a, 121b, 121c can include a dimension from about 100 microns (μm) to about 5000 μm. For example, in some embodiments, the depth of the well 122a, 122b, 122c can include a dimension of 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm, 1000 μm, 1500 μm, 2000 μm, 2500 μm, 3000 μm, 3500 μm, 4000 μm, 4500 μm, 5000 μm, any dimension or ranges of dimensions encompassed within the range of from about 100 μm to about 5000 μm.

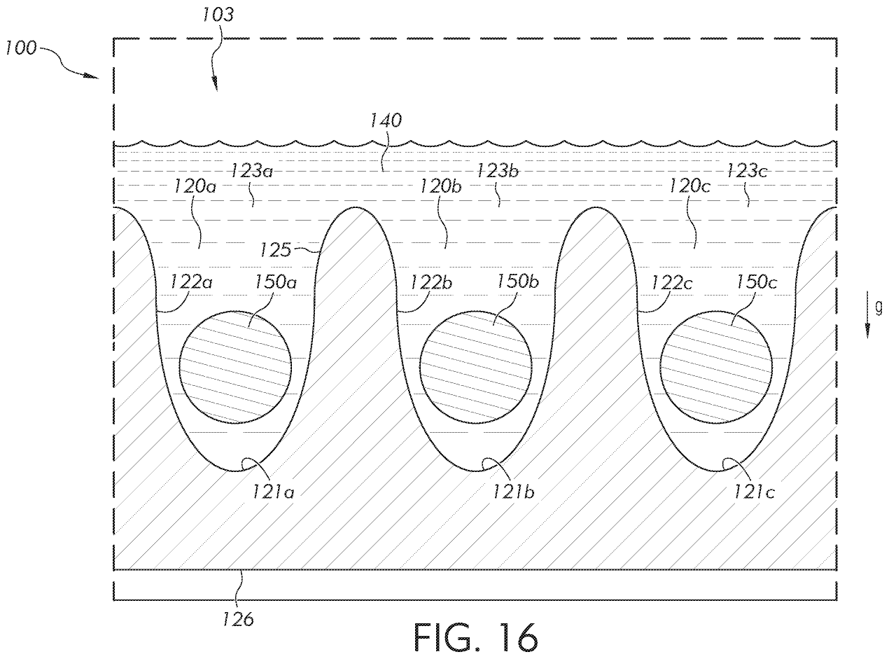

In some embodiments, three-dimensional cells 150 (e.g., spheroids, organoids 150a, 150b, 150c) (See FIG. 16) that can be cultured in at least one microcavity 120a, 120b, 120c of the plurality of microcavities 115 can include a dimension (e.g., diameter) of from about 50 μm to about 5000 μm, and any dimension or ranges of dimensions encompassed within the range of from about 50 μm to about 5000 μm. In some embodiments, dimensions greater than or less than the explicit dimensions disclosed can be provided and, therefore, unless otherwise noted, dimensions greater than or less than the explicit dimensions disclosed are considered to be within the scope of the disclosure. For example, in some embodiments, one or more dimensions of the opening 123a, 123b, 123c, the depth of the well 122a, 122b, 122c, and the dimension of the three-dimensional cells 150 (e.g., spheroids 150a, 150b, 150c) can be greater than or less than the explicit dimensions disclosed without departing from the scope of the disclosure.

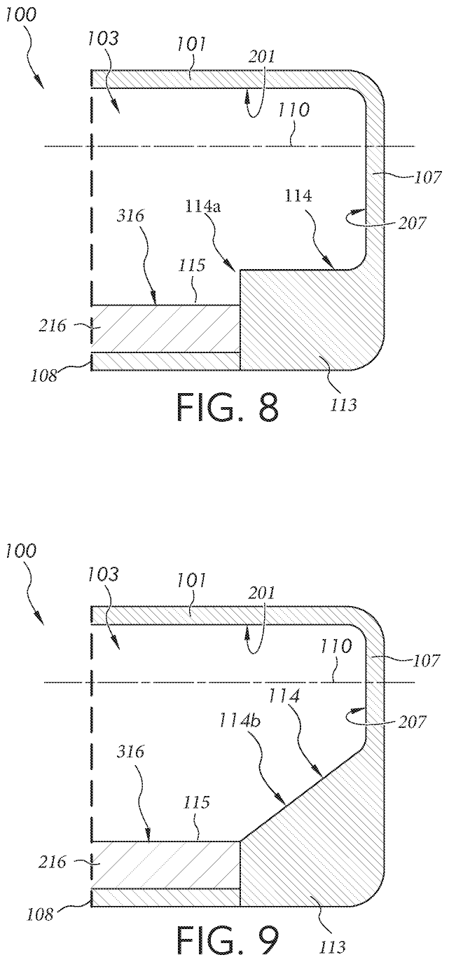

In addition or alternatively, as shown in FIG. 8, which shows a partial cross-sectional view of a portion of the vessel 100 along line 8-8 of FIG. 2, in some embodiments, a baffle 113 may be present along the end wall 107 of the vessel 100. In embodiments, such as that shown in FIG. 8, the baffle may have a stepped or square profile 114a. Likewise, as shown in FIG. 9, which shows an alternative exemplary embodiment of the partial cross-sectional view of the portion of the vessel 100 of FIG. 8, in some embodiments, the baffle 113 can include an inclined or angled profile 114b. As discussed more fully below, in some embodiments, a stepped or inclined baffle 113 can provide advantages with respect to methods of culturing cells in the cell culture vessel 100.

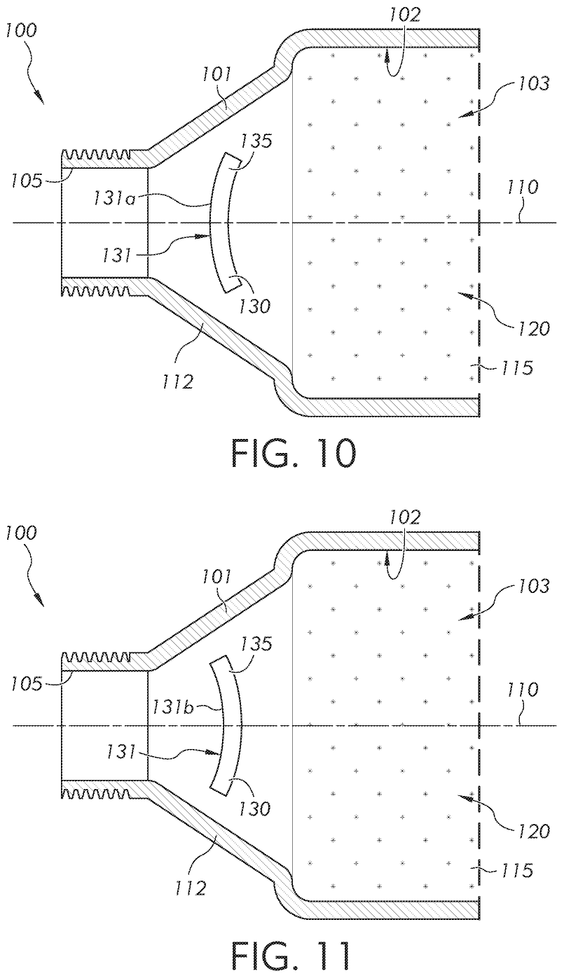

In addition or alternatively, turning back to FIG. 3 and FIG. 4, in some embodiments, the cell culture vessel 100 can include a dam 130 extending from the interior surface 212 of the neck 112 of the vessel 100. As shown in FIG. 3 and FIG. 4, the dam can be rectangular in shape. In some embodiments, the dam 130 can include a port-facing surface 131 obstructing a fluid path defined between the port 105 and the surface having a microcavity array 115. In some embodiments, the port-facing surface 131 of the dam 130 can be substantially perpendicular to the axis 110 of the vessel 100. Alternatively, as shown in FIG. 10, which shows an exemplary embodiment of a partial cross-sectional view of a portion of the first exemplary cell culture vessel 100 along line 10-10 of FIG. 1, in some embodiments, the port-facing surface 131 of the dam 130 can include a convex profile 131a. Additionally, as shown in FIG. 11, in some embodiments, the port-facing surface 131 of the dam 130 can include a concave profile 131b. In some embodiments, the dam 130 can be provided to obstruct flow of material into and out of the vessel 100. In additional embodiments, the dam 130 may be square or curved or any shape.

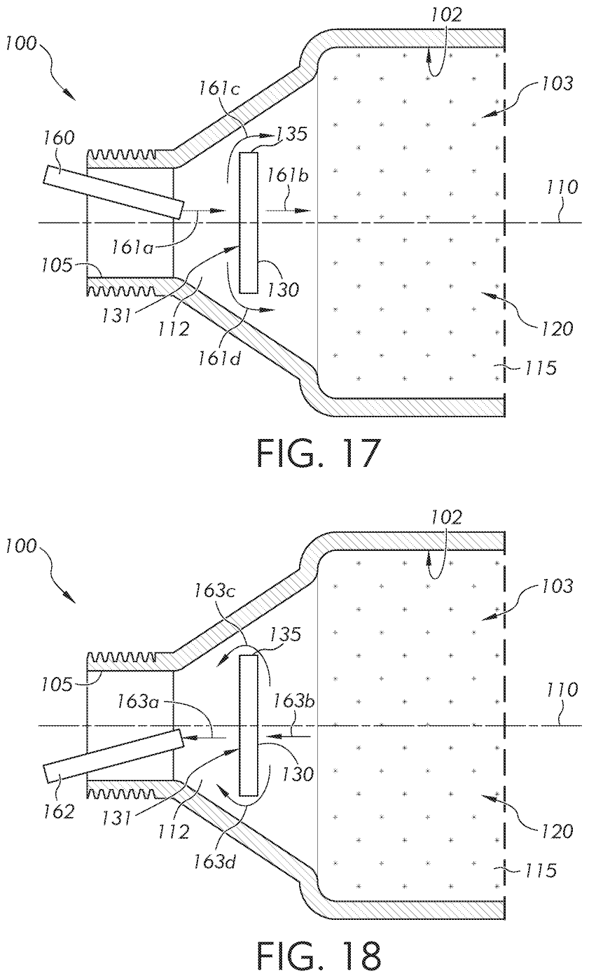

Moreover, turning back to FIG. 3, in some embodiments, at least a portion of an edge 135 of the dam 130 can be spaced a distance “dl” from the inner surface 201 of the top 101, for example. In some embodiments, by spacing at least a portion of a free end 135 of the baffle 130 from the inner surface 201, in some embodiments, access to a rear portion of the vessel 100 (e.g., opposite the port 105) can be provided. For example, in some embodiments, one or more instruments (not shown) can be inserted into the port 105 of the vessel 100 past the dam 130 (e.g., through the distance “d1”) to access a region of the cell culture chamber 103 positioned behind the dam 130. Accordingly, in some embodiments, the dam 130 can slow a velocity of the material flowing along at least one of a first flow path (see FIGS. 17 161a, 161b, and 161c) and a second flow path (see FIG. 18 163a, 163b) while also permitting bulk access into the cell culture chamber 103 of the vessel 100.

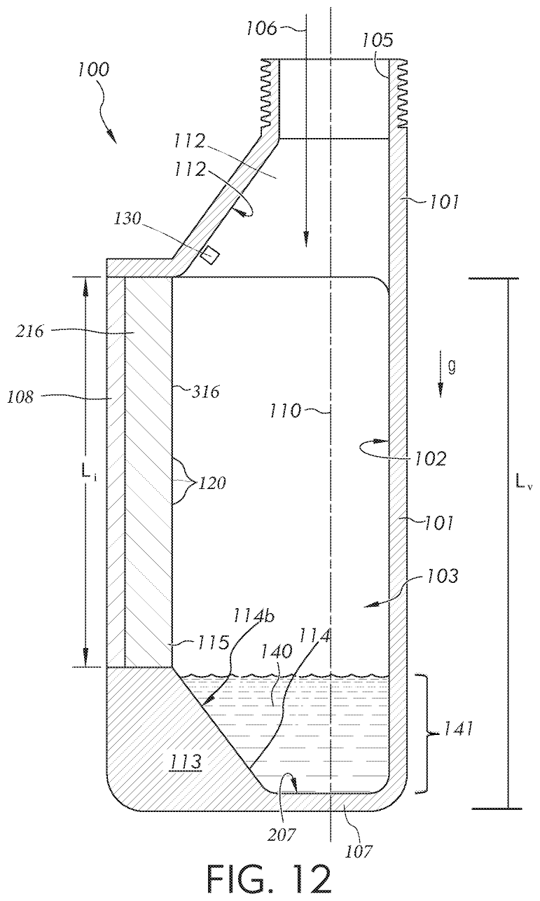

As shown in FIG. 12, in some embodiments, when the vessel 100 stands on the end wall 107, the axis 110 of the vessel 100 can extend substantially in the direction of gravity “g” while containing a predetermined amount of liquid 140 in the reservoir 141 of the cell culture chamber 103 without liquid of the predetermined amount of liquid 140 contacting one or more microcavities 120 of the plurality of microcavities 115. The reservoir 141 is the region between the top 101 and the baffle 113, bounded on the bottom by the end wall 107 and on the sides by the side walls 106 of the vessel 100. The reservoir 141 is structured and arranged to contain liquid without the liquid entering microcavities 120. There is a reservoir 141 in the vessel 100 because of the baffle 113, and because the array of microcavities 115 does not extend across the bottom 108 of the vessel all the way to the end wall 107, but instead the array of microcavities 115 ends at the baffle 113. That is, there is a reservoir 141 present in the vessel because the length of the array of microcavities (Lᵢ) is smaller than the length of the vessel (Lc). In addition, the reservoir 141 is sized and shaped to contain a predetermined amount of liquid 140.

For example, in some embodiments, the vessel 100 can be placed on, for example, a horizontal surface (not shown), resting on the end wall 107 with the axis 110 of the vessel 100 extending substantially upright in the direction of gravity “g”. In addition or alternatively, in some embodiments, the vessel 100 can be supported (e.g., held, suspended) by one or more structures (e.g., frame, mount, human hand, etc.) with the axis 110 extending substantially in the direction of gravity “g”. In some embodiments, the vessel 100 can be provided with the axis 110 extending substantially in the direction of gravity “g” based at least on one or more of positioning and supporting the vessel 100 while at least one of passing liquid (e.g., represented by arrow 106) through the port 105 from outside the vessel 100 into the cell culture chamber 103 and containing the predetermined amount of liquid 140 in the reservoir 141 of the cell culture chamber 103 without liquid of the predetermined amount of liquid 140 contacting one or more microcavities 120 of the array of microcavities 115.

In some embodiments, the predetermined amount of liquid 140 can be contained in the reservoir 141 of the cell culture chamber 103 without liquid of the predetermined amount of liquid 140 contacting one or more microcavities 120 of the plurality of microcavities 115 while the vessel 100 is stationary. Alternatively, in some embodiments, the predetermined amount of liquid 140 can be contained in the reservoir 141 of the cell culture chamber 103 without liquid of the predetermined amount of liquid 140 contacting one or more microcavities 120 of the plurality of microcavities 115 while the vessel 100 is in motion (e.g., not stationary). For example, in some embodiments, at least one of a translational motion and a rotational motion can be imparted on the vessel 100 while containing the predetermined amount of liquid 140 in the reservoir 141 of the cell culture chamber 103 without liquid of the predetermined amount of liquid 140 contacting one or more microcavities 120a, 120b, 120c of the plurality of microcavities 120. Thus, in addition to or alternative to extending substantially in the direction of gravity “g”, in some embodiments, the axis 110 of the vessel 100 can extend in one or more directions defining a non-zero angle relative to the direction of gravity “g” while containing the predetermined amount of liquid 140 in the reservoir 141 of the cell culture chamber 103 without liquid of the predetermined amount of liquid 140 contacting one or more microcavities 120 of the array of microcavities 115.

Moreover, in some embodiments, the orientation of the axis 110 of the vessel 100 (e.g., relative to the direction of gravity “g”) while containing the predetermined amount of liquid 140 in the reservoir 141 of the cell culture chamber 103 without liquid of the predetermined amount of liquid 140 contacting one or more microcavities 120 of the array of microcavities 115 can remain unchanged during a duration of time while containing the predetermined amount of liquid 140 in the reservoir 141 of the vessel 100. Alternatively, in some embodiments, the orientation of the axis 110 of the vessel 100 (e.g., relative to the direction of gravity “g”) while containing the predetermined amount of liquid 140 in the reservoir 141 of the cell culture chamber 103 without liquid of the predetermined amount of liquid 140 contacting one or more microcavities 120 of the plurality of microcavities 120 can change one or more times during a duration of time while containing the predetermined amount of liquid 140 in the reservoir 141 of the vessel 100. Moreover, in some embodiments, the predetermined amount of liquid 140 can be contained in the reservoir 141 of the vessel 100 without liquid of the predetermined amount of liquid 140 contacting one or more microcavities 120 of the plurality of microcavities 120 for an instant in time (e.g., as compared to a duration of time) in accordance with embodiments of the disclosure. In the embodiment shown in FIG. 12, the baffle face 114 is an inclined or angled baffle face 114b. While an insert 216 having an interior surface 316 having an array of microcavities 115 is illustrated in FIG. 12, one of ordinary skill in the art will recognize that the bottom 108 of the cell culture vessel having an integral array of microcavities 115 may also be provided.

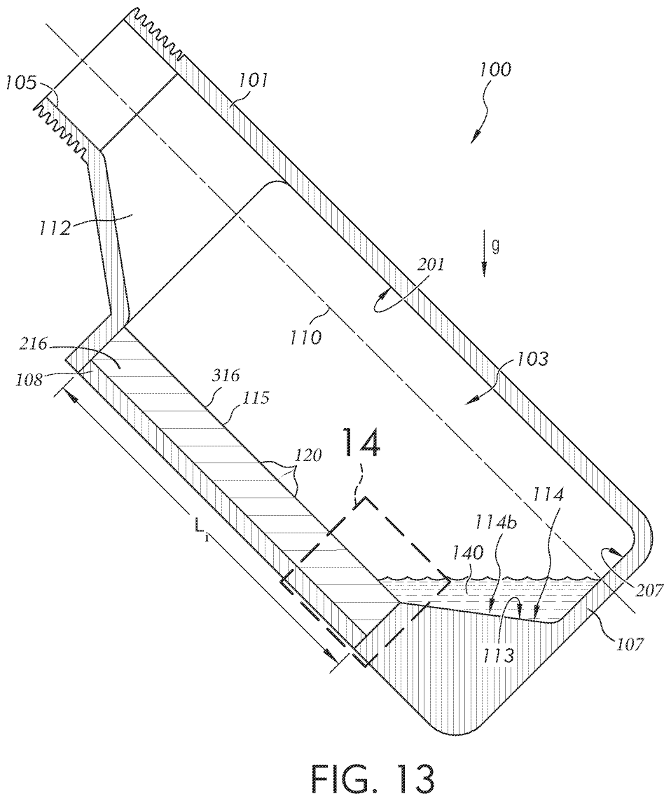

As shown schematically in FIG. 13, in some embodiments, the method can include moving the vessel 100 after containing the predetermined amount of liquid 140 in the reservoir 141 of the cell culture chamber 103 without liquid of the predetermined amount of liquid 140 contacting one or more microcavities 120 of the plurality of microcavities 115 (See FIG. 12) to cause at least a portion of the predetermined amount of liquid 140 to flow from the reservoir 141 over the surface having a microcavity array 115 along the length “Lᵢ” of the cell culture chamber 103 and deposit in at least one microcavity 120 of the plurality of microcavities 115 (or an insert 216 having an interior surface 316 having an array of microcavities 115). For example, in some embodiments, moving the vessel 100 can include at least one of translating and rotating the vessel 100 from a first orientation (e.g., the orientation provided in FIG. 12) to a second orientation (e.g., the orientation provided in FIG. 13). By moving the vessel 100 to cause at least a portion of the predetermined amount of liquid 140 to flow from the reservoir 141 over the surface having a microcavity array 115 along the length “L” of the cell culture chamber 103 and deposit in at least one microcavity 120 of the plurality of microcavities 115, the deposition of the liquid into the at least one microcavity 120 can be controlled and, in some embodiments, ensured.

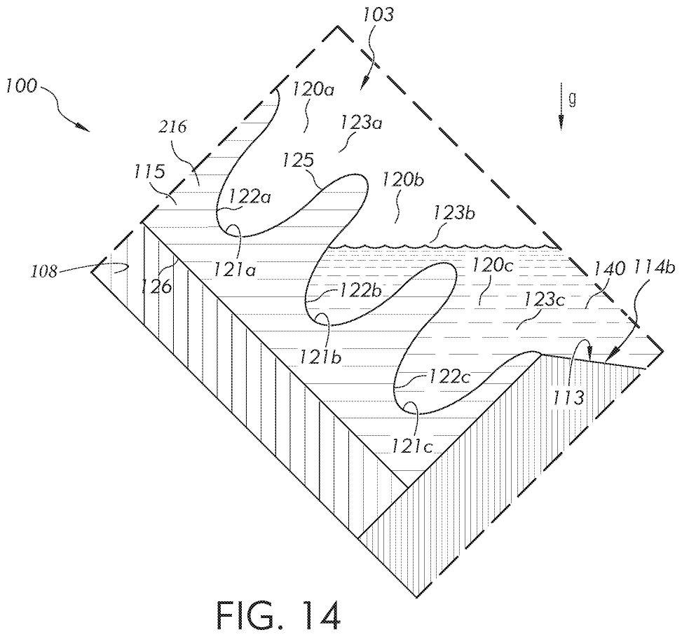

For example, FIG. 14 illustrates an enlarged schematic representation of an exemplary embodiment of a portion of the first exemplary cell culture vessel 100 taken at view 14 of FIG. 13 showing at least a portion of the predetermined amount of liquid 140 flowing from the reservoir 141 over the surface having a microcavity array 115 along the length “Lᵢ” of the cell culture chamber 103 and depositing in at least one microcavity 120 of the plurality of microcavities 115. FIG. 14 illustrates, the openings of the microcavities (123a, 123b, 123c), the wells of the microcavities (122a, 122b, 122c), a microcavity wall 125, and the bottom surface of the microcavities (121a, 121b and 121c). These features make up the microcavities (120a, 120b and 120c). In some embodiments, the movement of the vessel 100 to cause the liquid to flow can be controlled and slow (e.g., performed during a duration of time on the order of minutes). For example, it has been observed that directly filling the microcavities 120a, 120b, 120c of the plurality of microcavities 120 with liquid (e.g., not based on the method of the disclosure) can result in undesirable fill characteristics that at least one or inhibit or prevent cell growth. Without intending to be bound by theory, it is believed, when directly and quickly (e.g., performed during a duration of time on the order of seconds) attempting to fill the microcavities 120a, 120b, 120c of the plurality of microcavities 120 with liquid, that based at least on the surface tension of the liquid and the presence of gas within the well 122a, 122b, 122c that the liquid can form a barrier extending across the opening 123a, 123b, 123c of the microcavity 120a, 120b, 120c, thereby trapping gas (e.g., air or a bubble) within the wells 122a, 122b, 122c. In some embodiments, a rate of gas-permeation through the surface having a microcavity array 115 can be too slow (e.g., occurring for a duration of time on the order of hours and days) relative to a cell culture time that, for practical applications, the gas bubble remains within the well, 122a, 122b, 122c and the liquid remains outside the well 122a, 122b, 122c to the extent that cells are unable to be cultured within the well 122a, 122b, 122c. That is, bubbles are bad for cell culture.

However, by employing one or more features of the method of the disclosure, it has been observed that, for example, by moving the vessel 100 to cause at least a portion of the predetermined amount of liquid 140 to flow from the reservoir 141 over the surface having a microcavity array 115 along the length “Lᵢ” of the cell culture chamber 103 and deposit in at least one microcavity 120a, 120b, 120c of the plurality of microcavities 115, that liquid can enter the well 122a, 122b, 122c through a portion of the respective opening 123a, 123b, 123c of the at least one microcavity 120a, 120b, 120c. For example, as the liquid flows into the well 122a, 122b, 122c, the liquid can displace the gas within the well 122a, 122b, 122c, thereby filling the well 122a, 122b, 122c with the liquid. Moreover, in some embodiments, by performing the step during a duration of time on the order of minutes, for example, substantially all of the gas within the well 122a, 122b, 122c can be displaced from the well 122a, 122b, 122c as the liquid gradually flows from the reservoir 141 over the surface having a microcavity array 115 along the length “Lᵢ” of the cell culture chamber 103 and enters the well 122a, 122b, 122c through a portion of the respective opening 123a, 123b, 123c of the at least one microcavity 120a, 120b, 120c. That is, filling the microcavities 120 with liquid media slowly, from the reservoir 141, reduces the formation of bubbles in the microcavities 120 and improves cell culture.

In some embodiments, the profile 114 of the baffle 113 of the vessel 100 can provide a surface that facilitates the flow of the liquid from the reservoir 141 over the surface having a microcavity array 115 based at least on the movement of the vessel 100. For example, in some embodiments, the inclined or angled profile 141b of the baffle 113 can provide a sloped surface along which the fluid can flow from the reservoir 141 to the surface having a microcavity array 115 (the microcavity array can be provided by an insert 216). In some embodiments, the baffle 113 can abut the surface having a microcavity array 115 and fluid can flow from the reservoir 141 along the inclined profile 141b of the baffle 113 and deposit into at least one microcavity 120a, 120b, 120c with controlled flow (e.g., reduced or no liquid splashing and reduced or no turbulent flow), thereby providing a steady flow of liquid depositing into the wells 122a, 122b, 122c through the respective opening 123a, 123b, 123c of the at least one microcavity 120a, 120b, 120c while displacing gas from the well 122a, 122b, 122c.

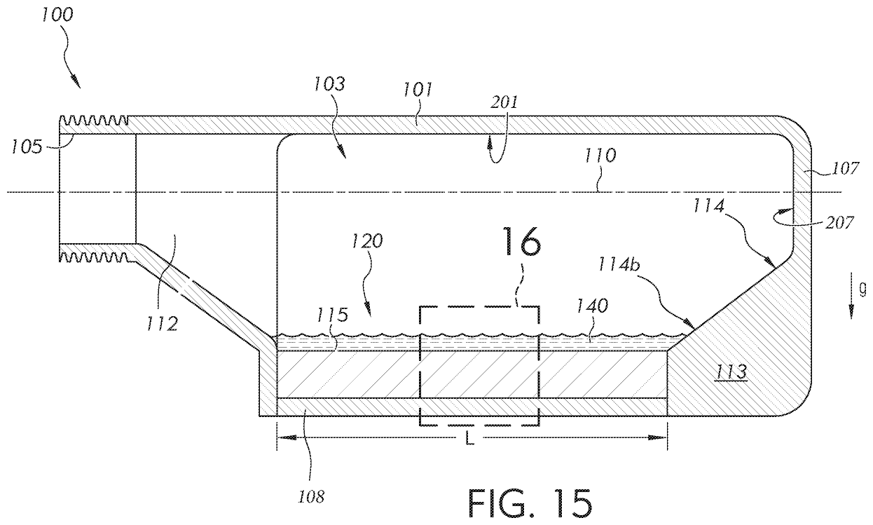

As shown in FIG. 15, in some embodiments, the predetermined amount of liquid 140 can be caused to flow from the reservoir 141 over the entire surface having a microcavity array 115 based at least on the movement of the vessel 100. Additionally, FIG. 16 illustrates an enlarged schematic representation of an embodiment of a portion of the cell culture vessel 100 taken at view 16 of FIG. 15 including a method of culturing cells 150 in the cell culture vessel 100. For example, in some embodiments, the method can include culturing cells 150 (e.g., spheroid 150a, spheroid 150b, spheroid 150c) in the at least one microcavity 120a, 120b, 120c of the plurality of microcavities 115 after depositing the at least a portion of the predetermined amount of liquid 140 in the at least one microcavity 120a, 120b, 120c. As shown in FIG. 15 and FIG. 16, in some embodiments, the axis 110 of the vessel 100 can be substantially perpendicular relative to the direction of gravity “g” while culturing cells 150 in the at least one microcavity 120a, 120b, 120c of the plurality of microcavities 120.

For example, as shown in FIG. 17, in some embodiments, a method of culturing cells 150 in the cell culture vessel 100 can include adding material (e.g., food, nutrients, liquid media) into the cell culture chamber 103 by inserting a dispensing-port 160 into the port 105, and then dispensing material from the dispensing-port 160 into the cell culture chamber 103. For example, in some embodiments, the method can include inserting the dispensing-port 160 into the port 105 of a vessel 100. The method can further include flowing material along a first flow path 161a, 161b in the cell culture chamber 103 of the vessel 100. The material can flow along the first flow path 161a, 161b by dispensing material from the dispensing-port 160, thereby adding material from outside the vessel 100 into the cell culture chamber 103. Additionally, in some embodiments, the method can include obstructing the flow of material along the first flow path 161a, 161b. For example, in some embodiments, the obstructing the flow along the first flow path 161a, 161b can include diverting the flow along the first flow path 161a, 161b with the dam 130. In some embodiments, the diverting the flow along the first flow path 161a, 161b with the dam 130 can include separating the flow along the first flow path 161a, 161b into at least two diverging flows 161c, 161d. For example, in some embodiments, at least one of the two diverging flows 161c, 161d can flow within the cell culture chamber 103 laterally around an outer perimeter of the dam 130 (e.g., between the outer perimeter of the dam 130 and the inner surface 102 of the sidewall 201). In some embodiments, the method can include culturing cells 150 in at least one microcavity 120a, 120b, 120c of the plurality of microcavities 120 (See FIG. 16) while dispensing material from the dispensing-port 160 into the cell culture chamber 103.

Additionally, as shown in FIG. 18, in some embodiments, the method can include removing material (e.g., waste, byproduct, liquid media) from the cell culture chamber 103 by inserting a collecting-port 162 through the port 105, and then collecting material from the cell culture chamber 103 with the collecting-port 162. For example, in some embodiments, the method can include inserting the collecting-port 162 into the port 105, flowing material along a second flow path 163a, 163b in the cell culture chamber 103 by collecting material with the collecting-port 162, thereby removing material from the cell culture chamber 103. In some embodiments, the method can include obstructing the flow of material along the second flow path 163a, 163b. For example, in some embodiments, the obstructing the flow along the second flow path 163a, 163b can include diverting the flow along the second flow path 163a, 163b with the dam 130. In some embodiments, the diverting the flow along the second flow path 163a, 163b with the dam 130 can include separating the flow along the second flow path 163a, 163b into at least two diverging flows 163c, 163d. For example, in some embodiments, at least one of the two diverging flows 163c, 163d can flow within the cell culture chamber 103 laterally around an outer perimeter of the dam 130 (e.g., between the outer perimeter of the baffle 130 and the inner surface 102 of the side wall 106). In some embodiments, the method can include culturing cells 150 in at least one microcavity 120a, 120b, 120c of the plurality of microcavities 120 while collecting material from the cell culture chamber 103 with the collecting-port 162.

In some embodiments, obstructing the flow of material along at least one of the first flow path 161a, 161b and the second flow path 163a, 163b with the dam 130 while culturing cells 150 in at least one microcavity 120a, 120b, 120c of the plurality of microcavities 120 can respectively add and remove material from the cell culture chamber 103 of the vessel 100 without, for example, interfering with the culturing of the cells 150. For example, in some embodiments, the dispensing-port 160 can add material to the cell culture chamber 103 by flowing (e.g., dispensing, blowing) the material from the dispensing-port 160 into the cell culture chamber 103 with a velocity along the first flow path 161a, 161b, thereby creating a positive pressure force in and around the port 105 and the cell culture chamber 103. Likewise, in some embodiments, the collecting-port 162 can remove material from the cell culture chamber 103 by flowing (e.g., collecting, aspirating) the material from the cell culture chamber 103 into the collecting-port 162 with a velocity along the second flow path 163a, 163b, thereby creating a negative pressure force in and around the port 105 and the cell culture chamber 103. Accordingly, in some embodiments, the dam 130 can slow a velocity of the material flowing along at least one of the first flow path 161a, 161b and the second flow path 163a, 163b, thereby respectively decreasing the positive pressure force and the negative pressure force in and around the port 105 and the cell culture chamber 103. In some embodiments, the dam 130 can, therefore, at least one of reduce and prevent the flow of material along at least one of the first flow path 161a, 161b and the second flow path 163a, 163b from dislodging cells 150 being cultured in at least one microcavity 120a, 120b, 120c of the plurality of microcavities 120. For example, in some embodiments, if flow of material dislodges one or more cells, one or more microcavities 120a, 120b, 120c can include more than one spheroid or no spheroids. Additionally, in some embodiments, by at least one of reducing and preventing the flow of material from dislodging cells 150 being cultured in the vessel 100, better quality cell cultures and more accurate scientific results relating to the cell cultures can be obtained.

Methods of culturing cells in the first exemplary cell culture vessel 100 will now be described with reference to FIGS. 12-16. As shown in FIG, 12, in some embodiments, a method of culturing cells 150 (See FIG. 16) in the cell culture vessel 100 can include passing liquid (e.g., represented by arrow 106) through the port 105 from outside the vessel 100 into the cell culture chamber 103, thereby providing a predetermined amount of liquid 140 in the cell culture chamber 103. Although the method is described with respect to the vessel 100 including the inclined profile 114b of the baffle 113, it is to be understood that, in some embodiments, the method can be employed in a same or similar manner with respect to the non-planar boundary portion 114 (shown in FIG. 3), and the stepped profile 114a (shown in FIG. 8), as well as other profiles of the inner surface 102 of the wall 101 of the vessel 100 in accordance with embodiments of the disclosure, without departing from the scope of the disclosure.

In some embodiments, the method can include containing the predetermined amount of liquid 140 in a reservoir 141 of the cell culture chamber 103 without liquid of the predetermined amount of liquid 140 contacting one or more microcavities 120a, 120b, 120c of the plurality of microcavities 120 of the surface having a microcavity array 115. For example, in some embodiments, liquid of the predetermined amount of liquid 140 can contact the baffle 113 while containing the predetermined amount of liquid 140 in the reservoir 141 of the cell culture chamber 103 without liquid of the predetermined amount of liquid 140 contacting one or more microcavities 120a, 120b, 120c of the plurality of microcavities 120. As discussed more fully below, preventing liquid of the predetermined amount of liquid 140 from contacting one or more microcavities 120a, 120b, 120c of the plurality of microcavities 120, at this stage of the method, can provide several advantages that, for example, facilitate improved culturing of the cells 150 (See FIG. 16).

As shown in FIG. 3, which shows a cross-sectional view of the vessel 100 along line 3-3 of FIG. 2, bottom 108 may have an insert 216 resting upon bottom 108. Insert 216 has an inner surface 316. The inner surface 316 of insert 216 has an array of microcavities 115 in additional embodiments, the cell culture vessel 100 can include a surface having a microcavity array 115 including a plurality of microcavities 120 (See FIGS. 5-7). In some embodiments, the surface having a microcavity array 115 and the inner surface 102 of the wall 101 can define a cell culture chamber 103 of the vessel 100, with a port 105 extending through the wall 101 in fluid communication with the cell culture chamber 103. For example, in some embodiments, the cell culture chamber 103 can include an internal spatial volume of the vessel 103.

In embodiments, the interior surface of the necked opening may have one or more dams to divert the flow of liquid into the vessel and to reduce turbulence experienced by cells cultured on the cell culture surface when liquid is introduced into the vessel. In additional embodiments, a method of culturing cells can include changing media by first placing the vessel so that it sits with the necked opening facing up, allowing fluid to enter the vessel by flowing the fluid along the top of the vessel, and removing fluid from the back end of the vessel. In further embodiments, a method of culturing cells can include introducing media while the vessel is placed so that it sits with the necked opening facing up, allowing media to fill the back end of the vessel, then allowing media to flow onto the surface having an array of microcavities by carefully tilting the vessel until that the surface having an array of microcavities is down.

In some embodiments, a vessel has a dam in the neck of the vessel to interrupt the flow of liquid into the cell culture chamber. In additional embodiments, a method of culturing cells can include inserting a dispensing-port into the port of the vessel. The method can include flowing material along a first flow path in a cell culture chamber of the vessel defined by an inner surface of the wall and a surface having a microcavity array, thereby adding material from outside the vessel into the cell culture chamber. The method can include obstructing the flow of material along the first flow path.

In some embodiments, a method of culturing cells can include passing liquid through a port in a vessel from outside the vessel into a cell culture chamber of the vessel defined by an inner surface of the wall and a surface having a microcavity array, thereby providing a predetermined amount of liquid in a reservoir of the cell culture chamber. The method can include containing the predetermined amount of liquid in the reservoir of the cell culture chamber without the liquid contacting one or more of the plurality of microcavities.

In some embodiments, a cell culture vessel can include a wall and a surface having an array of microcavities. The surface having a microcavity array and the inner surfaces of the walls and the top of the vessel defines a cell culture chamber of the vessel, or the cell culture chamber. A port can extend through a wall of the vessel in fluid communication with the cell culture chamber.

A number of aspects of cell culture vessels and methods of culturing cells have been disclosed herein. A summary of some selected aspects is presented.

It will be apparent to those skilled in the art that various modifications and variations can be made to the present disclosure without departing from the spirit and scope of the disclosure. Thus, it is intended that the present disclosure cover the modifications and variations of this disclosure provided they come within the scope of the appended claims and their equivalents.

1. A cell culture vessel comprising:
a cell culture chamber comprising a top, a bottom, sidewalls and a necked opening extending through a wall of the cell culture chamber in fluid communication with the cell culture chamber;
a cell culture surface having an array of microcavities;
a baffle have in a baffle length (L_{b}) between a wall opposite the necked opening and the array of microcavities;
wherein the array of microcavities extends a Length (Lᵢ) which is less than a length of the cell culture chamber (L_{c}); and,
wherein the baffle length (L_{b}) plus the Length of the array of microcavities (Lᵢ) equals the length of the cell culture chamber (L_{c});
so that when the vessel is oriented so that the necked opening is up, there is a reservoir between the baffle and the top of the cell culture chamber.

2. The cell culture vessel of claim 1, wherein the bottom comprises the array of microcavities.

3. The cell culture vessel of claim 2, wherein the array of microcavities is integral to the interior surface of the bottom.

4. The cell culture vessel of claim 3, wherein the bottom surface of the array of microcavities is planar.

5. The cell culture vessel of claim 3, wherein the bottom surface of the array of microcavities comprises an array of microprojections.

6. The cell culture vessel of claim 1, further comprising an insert on the bottom, wherein the insert comprises the array of microcavities.

7. The cell culture vessel of claim 6, wherein the insert is affixed to the bottom.

8. The cell culture vessel of claim 6, wherein the bottom surface of the array of microcavities is planar.

9. The cell culture vessel of claim 6, wherein the bottom surface of the array of microcavities comprises an array of microprojections.

10. The cell culture vessel of claim 1, wherein the baffle is inclined.

11. The cell culture vessel of any one of claim 1, wherein the baffle is square.

12. The cell culture vessel of any one of claim 1, further comprising a dam in the necked opening of the vessel.

13. The cell culture vessel of claim 12, wherein the dam is curved.

14. The cell culture vessel of claim 12, wherein the dam is rectangular.

15. A method of culturing cells in a cell culture vessel according to any one of claim 1, comprising:
resting the vessel on the endwall opposite the necked opening of the vessel;
introducing cells suspended in liquid media into the vessel where the cells and liquid media are placed in the reservoir in the space between the baffle and the top of the vessel;
rotating the vessel so that the cells and liquid media flow onto the cell culture surface comprising the array of microcavities.

16. The method of claim 15, further comprising the step of culturing the cells in the vessel.

17. The method of claim 16, further comprising rotating the vessel so that the cells and media flow into the reservoir.

18. A method of culturing cells comprising:
inserting a dispensing-port into a necked opening of a cell culture vessel;
flowing material along a first flow path in a cell culture chamber of the vessel defined by an inner surface of the wall and a surface comprising a microcavity array by dispensing material from the dispensing-port, thereby adding material from outside the vessel into the cell culture chamber; and
obstructing the flow of material along the first flow path.

19. The method of claim 18, the obstructing the flow along the first flow path comprises diverting the flow along the first flow path with a dam.

20. The method of claim 19, the diverting the flow along the first flow path with the dam comprises separating the flow along the first flow path into at least two diverging flows.

21. The cell culture vessel of claim 3, wherein the baffle is inclined.

22. The cell culture vessel of claim 5, wherein the baffle is inclined.

23. The cell culture vessel of claim 6, wherein the baffle is inclined.

24. The cell culture vessel of claim 3, wherein the baffle is square.

25. The cell culture vessel of claim 5, wherein the baffle is square.

26. The cell culture vessel of claim 6, wherein the baffle is square.

27. The cell culture vessel of claim 9, wherein the baffle is square.

28. The cell culture vessel claim 3, further comprising a dam in the necked opening of the vessel.

29. The cell culture vessel claim 5, further comprising a dam in the necked opening of the vessel.

30. The cell culture vessel claim 6, further comprising a dam in the necked opening of the vessel.

31. The cell culture vessel claim 9, further comprising a dam in the necked opening of the vessel.

32. The cell culture vessel claim 11, further comprising a dam in the necked opening of the vessel.
